(19) United States
(12) Patent Application Publication
(10) Pub. No.: US 2007/0073283 A1
(43) Pub. Date: Mar. 29, 2007

(54) Medical energy manipulation device and method

(76) Inventor: David William Gomes, Jamaica Plain, MA (US)

Correspondence Address: David W. Gomes, 37 Slocum Road, Jamaica Plain, MA, 02130, US

(21) Appl. No.: 11/292,453

(22) Filed: Dec. 2, 2005

Related U.S. Application Data
(60) Provisional application No. 60/721,166, filed on Sep. 28, 2005.

(51) Int. Cl. A61B 18/04

(52) U.S. Cl. 606/32

(57) ABSTRACT

An energy manipulation device and method provide a figurine representing at least a portion of a living body, one or more energy conduction elements located on or within the figurine to apply energy to predetermined energy flow locations of the living body; and circuitry adapted for energizing the energy conduction elements.

### RELATED APPLICATIONS

The present application is related to and claims priority from U.S. Provisional Application Ser. No. 60/721,166, filed Sep. 28, 2005.

### FIELD OF THE INVENTION

The present invention generally relates to energy healing devices, and in particular to such devices which treat the energy field of a patient rather than directly treating the patient.

### BACKGROUND OF THE INVENTION

Energy healing has been practiced for thousands of years. Ancient Chinese culture developed the theory that energy flow within the body determines a person's health, and this theory became the basis of Chi Gong (or Qigong) healing and other forms of treatment such as Tuina massage and acupuncture. These direct physical treatments involve the application of energy to specific points on the body that are part of a system of meridians and vessels along which chi, or bodily energy is thought to flow, with the intent of manipulating that flow. The general effect of energy healing is to stimulate the natural healing processes of the subject being treated. The bodily energy flow being manipulated is too subtle for measurement by modern science, and the meridians themselves have no corporal existence within the body. The energy applied to the body may be in the form of physical pressure, the effect of acupuncture needles, with or without heat or electricity, or even subtle energy that is coupled to portions of a patient's body located in close proximity to, or touching the hands of a healer, as well as other forms.

The ancient chi gong and acupuncture work has been recently extended to cover distant energy healing and particularly by the work of Mr. Thomas Tam, a Licensed Acupuncturist in the Commonwealth of Massachusetts. Mr. Tam developed a practice, referred to as Tong Ren Therapy, in which a therapist holds a plastic acupuncture doll and repeatedly strikes the doll with a magnetic tipped hammer to stimulate healing energy flow within a patient. The doll is struck at selectable treatment points based upon the patient's diagnosis, in the same manner that the locations for acupuncture needles are determined. The therapist and subject are typically located in the same room, however, effective healing may be produced over greater distance. Effective healing is also achieved when the therapist and subject are telephonically linked. Tong Ren therapy is very effective when practiced in groups as described at “http://www.yinyanghouse.com/tongren/index.html”. The practice further works for self treatment and even by relatively untrained participants. The particular treatment points used have been developed by Mr. Tam during his many years of successful acupuncture practice and are published. These treatment points have been effective for remitting or improving the symptoms of many illnesses including serious illnesses such as cancer, Parkinson's disease, Multiple Sclerosis and Lou Gerhig's Disease (Amyotropic Lateral Sclerosis).

Mr. Tam has even further extended the practice to the use of a laser pointer aimed at the doll, wherein the doll is located in general proximity to one or more patients to distribute healing energy to those patients. The point of light produced by the laser is used to illuminate the above-mentioned treatment points corresponding to particular ailments.

In view of these advances and the success of Mr. Tam's energy healing, it is desirable to make the treatment simpler and more convenient for use by untrained subjects.

### SUMMARY OF THE INVENTION

In one embodiment, an energy manipulation device, comprises a figurine substantially shaped like at least a portion of a living body, one or more energy conduction elements located either on or within the figurine to apply an energy field to predetermined portions of the figurine corresponding to energy healing locations of the living body, and circuitry adapted for energizing the one or more energy conduction elements.

The energy manipulation device may further comprise separate illumination devices located at one or more of the predetermined portions of the figurine. The separate illumination devices may be light emitting diodes or laser diodes. The circuitry may be adapted to cause flashing of the separate illumination devices.

The circuitry may include a programmable control device adapted for determining energizing of the one or more energy conduction elements. The control device may be adapted to allow selection of a code corresponding to a particular ailment for causing activation of the energy conduction elements corresponding to treatment for the particular ailment. The control device may be adapted to cause sequential treatment of a plurality of ailments based upon initial selection of the plurality of ailments. The control device may be adapted to determine an energy waveform for energy flow along the one or more energy conduction elements.

The energy conduction elements may include electrical conductors located to create an electromagnetic field across the predetermined portions of the figurine. The electrical conductors may include a pair of conductors generally located on opposing sides of the predetermined portions of the figurine. The pair of electrical conductors may be generally located on opposing sides of a portion of the figurine representing a spine of the living body. The circuitry may be adapted to apply an AC voltage across the pair of electrical conductors. The figurine may be adapted to allow pins to pierce an outer surface thereof for concentrating electromagnetic energy at selectable points on or within the figurine.

The energy conduction elements may be routed through the figurine in accordance with portions of chi gong energy vessels or meridians of the living body. The figurine may be miniaturized.

In another embodiment, a method for performing energy healing on a subject, comprises the steps of locating a figurine representing at least a portion of a subject within proximity of the subject, and routing artificial energy flow within the figurine at locations corresponding to energy healing locations within the subject.

The step of routing may include conducting electrical or optical energy through the figurine. The method may further comprise the steps of repeating the steps of locating and routing with a specific subject over a period of time and observing any effects produced in the subject. The step of routing may be adapted to enhance physical or mental performance of the subject. The figurine may be substantially shaped like a human or animal subject.

### BRIEF DESCRIPTION OF THE DRAWINGS

The present invention is illustratively shown and described in reference to the accompanying drawings, in which:

FIG. 1 is a front representation of a human body;

FIG. 2 is a rear representation of the human body of FIG. 1;

FIG. 3 is a rear view of a figurine constructed in accordance with one embodiment of the present invention and showing energy treatment devices located either on or within the figurine;

FIG. 4 is a side schematic view of the figurine of FIG. 3;

FIG. 5 is a block circuit diagram of the control device of FIG. 3;

FIG. 6 is a table showing several illnesses and treatment codes for use by the embodiment of FIGS. 3 and 4;

FIG. 7 is a side schematic view of another figurine constructed in accordance with another embodiment of the present invention; and

FIG. 8 is a rear view of another figurine constructed in accordance with another embodiment of the present invention.

### DETAILED DESCRIPTION OF THE DRAWINGS

The methodology of the present embodiments involves manipulating artificial energy flow within a figurine representing at least a portion of a subject to be treated, to stimulate desired healing energy flow within the subject.

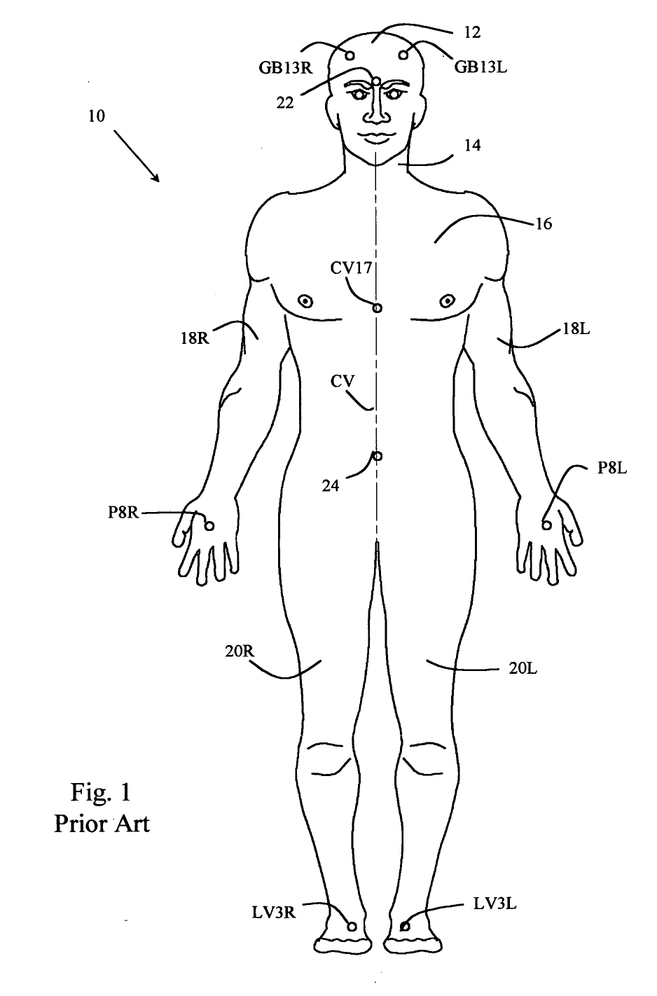

FIG. 1 shows a human body 10 including head 12, neck 14, torso 16, arms 18L, 18R and legs 20L, 20R. FIG. 1 further shows a number of common treatment points that are used for different types of energy healing, including GB13L, GB13R, Yintang 22, CV17, Dantian 24, P8L, P8R, LV3L, LV3R. These treatment points are indicated by circles. Still further represented in FIG. 1 is a portion of an energy vessel, the Conception Vessel CV.

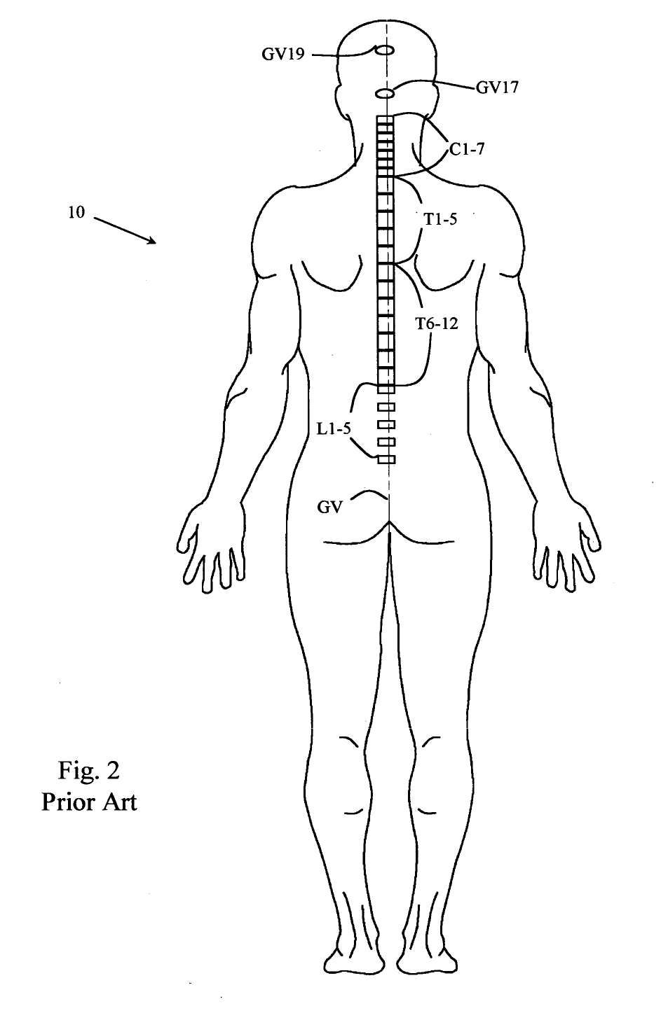

FIG. 2 is a view of the rear side of body 10, which view also shows various treatment points including the sensory cortex GV19, medulla GV17, cervical vertebrae C1-C7, thoracic vertebrae T1-T12, and lumbar vertebrae L1-L5. Also shown is the Governing Vessel GV. The spinal vertebrae shown are represented by rectangles and are not shown in phantom even though they are located within body 10.

The terms “treatment point(s)” or “energy healing point(s)” or “energy flow location(s)” or similar terms used herein in reference to the present embodiments are intended to include any specific portion of a living body to which energy healing may be applied to address a particular condition and are not intended to be limited to just formal treatment points known in acupuncture practice or other energy healing practice. For example, the healing of a broken bone may be expedited by using an embodiment of the present invention to apply energy to that portion of the figurine representing the location of the broken bone in a subject's body. Likewise, it may be desirable to enhance energy flow at any general location for non-healing purposes, such as stimulating hormone production or enhancing physical or mental performance of a subject. Thus, the “treatment point(s)” or “energy healing point(s)” or “energy flow location(s)” of the present invention do not need to be directly associated with a formal meridian, vessel or other chi gong treatment point.

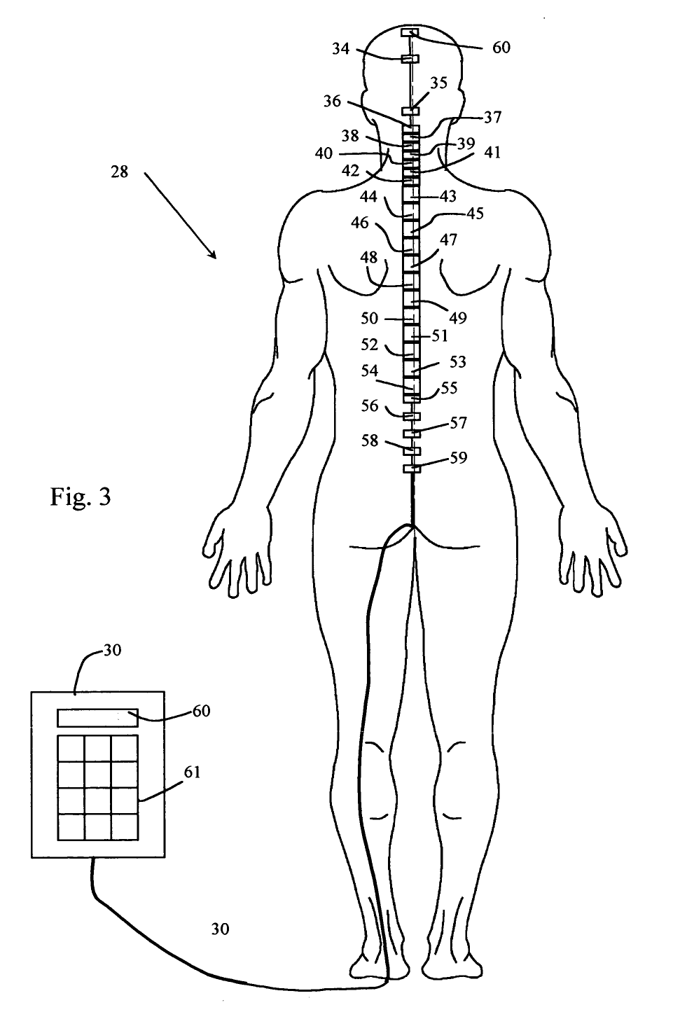

FIG. 3 is a rear view of a figurine 28 representing a human body and having circuitry adapted for applying energy to predetermined energy treatment points. FIG. 3 shows a control device 30 connected to figurine 28 by electrical conductors 32, which pass through leg 20L and are connected to illumination devices 34-59 located to correspond to treatment points GV19, GV17, C1-C7, T1-T12 and L1-L5 (FIG. 2), respectively. It is preferred that figurine 28 be generally shaped like a subject to be treated. Significant detail is not necessary. For example, limbs may or may not be touching the torso or each other, but non-touching limbs may be more effective. Also, use of an adult figurine 28 may be effective for treating an infant. The present embodiments should also work for treating animals, and a generally appropriate animal figurine is preferred.

Any suitable illumination devices may be used, such as light emitting diodes (LEDs) or laser diodes. Illumination devices 34-59, 62 are represented by rectangles and may be located either on the outside of or within figurine 28. Likewise, any suitable correspondence between illumination devices and treatment points may be used. For example, separate illumination devices may be used for each treatment point (as shown), multiple treatment points may be covered by a single illumination device, or individual treatment points may be covered by one or more adjacent illumination devices. Similarly, treatment of an area which is not a location for an illumination device may be accomplished by illuminating one or more adjacent devices.

Control device 30 includes a display device 60, such as an LCD, and an input keypad 61. Control device 30 is shown to be physically separate from figurine 28 for purposes of clarity, and may alternatively be integrally constructed with figurine 28 by forming a base (not shown) for figurine 28, or even located within the torso of figurine 28.

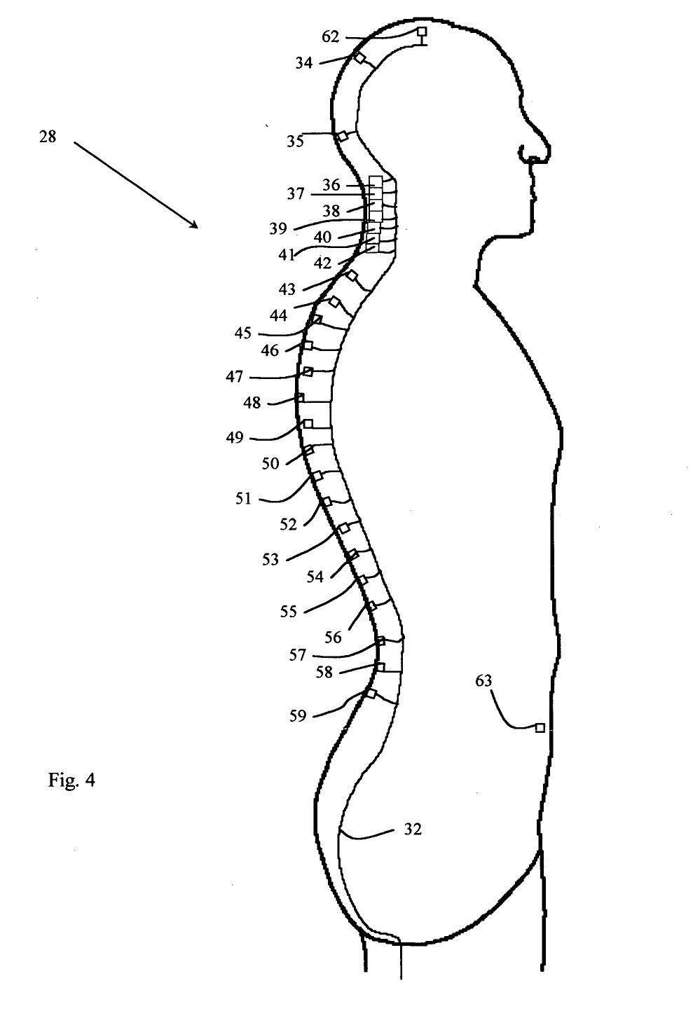

FIG. 4 is a side schematic view of a portion of figurine 28, wherein illumination devices and electrical conductors 32 are located within figurine 28. Illumination devices 34-59, 62 are represented as being located on an interior surface of figurine 28 and may also be held in place by any suitable construction. This may include integral construction with a rear wall of figurine 28. Thus, in one embodiment, illumination devices 34-59, 62 may be visible outside of figurine 28 to act as a teaching aid for the treatment points. An additional illumination device 62 is located at another treatment point called the motor cortex or GV20. Yet another illumination device 63 is nominally located at Dantian 24 (FIG. 1). Figurine 28 may include additional illumination devices, which also be selectively located anywhere within the volume of figurine 28.

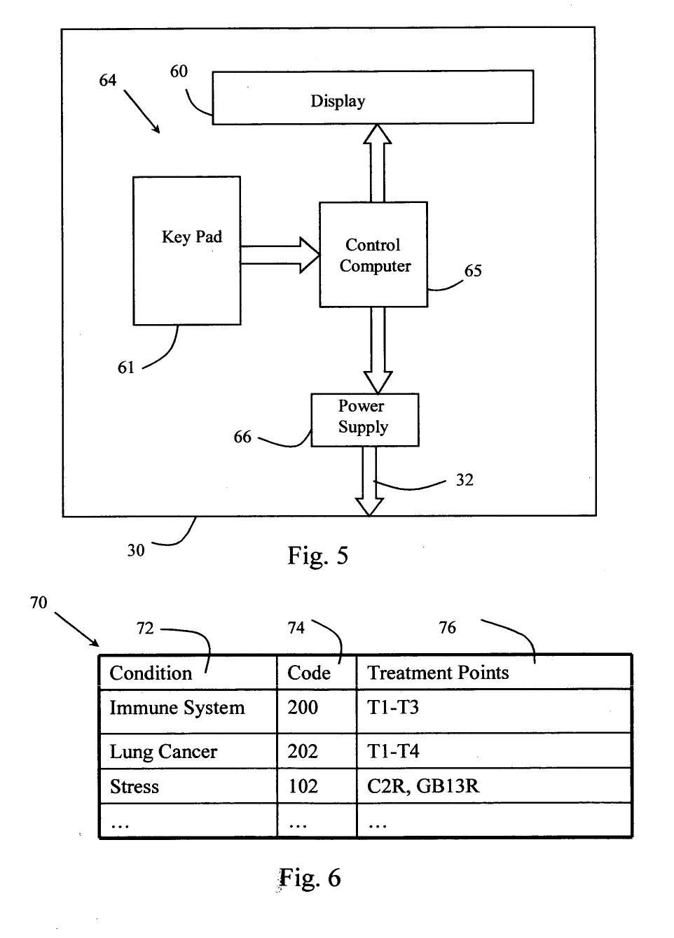

FIG. 5 shows a circuit diagram 64 including the components of control device 30, such as display 60 and keypad 61. Also shown are a control computer 65 and a power supply 66 coupled to electrical conductors 32. Control computer 65 includes suitable program instructions for prompting a user with display 60 to enter treatment instructions into keypad 61 and to correspondingly control power supply 66 to deliver appropriate energy waveforms to conductors 32.

In operation, control computer 65 uses display device 60 to prompt a person to enter one or more treatment codes into keypad 61. Control computer may also be adapted to prompt a person to set a treatment time corresponding to each treatment code. Control computer 65 may also accept multiple codes to enable the treatment of multiple ailments, either sequentially or simultaneously. The treatment times may be simple time periods or may be related to a clock time to cause treatments to occur at specific times. For example, figurine 28 may be used in a manner similar to an alarm clock to stimulate a person's energy just before it is time to wake up in the morning. It might further include some initial treatment at bedtime to reduce stress and a further overnight treatment to promote sleep. This routine could further be programmed to automatically repeat every night.

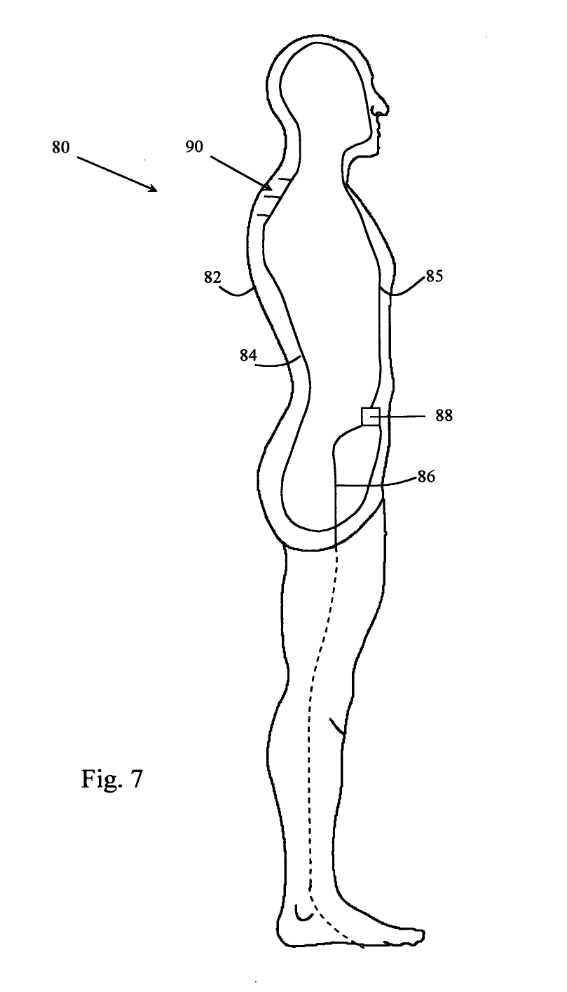

FIG. 6 is a table 70, which lists several conditions 72 or ailments that a person might wish to treat. Corresponding to each condition 72 is a treatment code 74 and a series of treatment points 76. A person would look up the condition to be treated and enter the respective treatment code 74 into key pad 61 (FIG. 5). In response, control computer 65 (FIG. 5) would cause the delivery of electrical energy to respective illumination devices located at treatment points 76. Although control computer 64 may be programmed to allow a list of treatment points to be directly entered, the use of treatment codes 74 is preferable for the convenience of the user, as multiple treatment points are typically more beneficial for each condition. Also, the different treatment points 76 for any particular illness may either be energized simultaneously, sequentially, or in any combination thereof, which is best suited for medical effectiveness. The programmed sequencing, or even singular illumination, of treatment points may also include programmed duration times for each illumination. Thus, the simple entry of a code can be used to execute a complex treatment protocol. Likewise, computer 65 may include a recording function to retain programmed sequences in memory.

It is also understood that the treatment programming may be determined separately from the figurine and then transferred to the figurine and used by a simple sequencer circuit to energize selected treatment points. Thus, the sequence may be determined in a separate computer and transferred to the figurine in any a suitable manner, such as with a flash memory device or through a communications link. There is also a growing area of remotely programmable medical devices, which are carried by patients for purposes of monitoring and drug administration control. The present embodiments are readily adaptable to that type of methodology.

It is further understood that the control function might also be radically minimized by constructing the illumination devices with integral on/off switches so that a user would manually switch on devices corresponding to an intended treatment points.

It is still further understood that figurine 28 may be dedicated to causing energy flow at, or illuminating a single location or a predetermined set of locations, thus reducing the function of control device 30 to an on/off control of that energy flow. Lastly, any combination of different control methods might be used.

Once control computer 65 is programmed, it is only necessary to locate figurine 28 in proximity to the subject to be treated to allow the energy flow within figurine 28 to have an effect upon the subject. For example, figurine 28 may be simply held by a subject or placed in contact with a subject. Such proximity may also include locating figurine 28 on a piece of furniture positioned next to a bed or chair in which the subject is located. It is understood that figurine 28 might be constructed with an integral control device and padded, like a teddy bear, to allow closer proximity to a sleeping subject, or a child in need of additional psychological comfort. Likewise, figurine 28 may be miniaturized to easily facilitate transport by a subject in any suitable manner, such as within a pocket or purse or on a lanyard around a subject's neck.

Although the embodiments described above refer to the treatment of specific ailments, figurine 28 may be alternately, or simultaneously controllable to provide generic treatments. For example, some energy healing methodologies relate ailments to specific spinal vertebrae, and each vertebra is related to specific bodily organs. So rather than treating for a specific ailment and being concerned with diagnosis and selection of treatment points, control device 30 may be adapted to provide a sequential illumination of the devices 36 to 54 (FIGS. 3, 4) to thereby simply stimulate energy flow through the spine and treat a long list of potential ailments in a prophylactic manner. Because this form of energy healing primarily stimulates the body's own self-healing capability, such a generic treatment might slow the effects of aging for an elderly subject. Likewise, such a generic form of treatment might be developed and used to stimulate physical performance in an athlete, or mental performance as desired. For example, athletic performance might be enhanced by energizing Dantian 24 (FIG. 1), which is an energy storage vessel. Likewise, mental performance might be stimulated by energizing C2 (FIG. 2; or LED 37, FIGS. 3, 4), GB13L, GB13R, and Yintang 22 (FIG. 1).

As mentioned, the subtle energy levels involved in this treatment cannot be measured. There is some speculation that the subject's energy field, or at least an electromagnetic component thereof, links to the figurine and is replicated thereon. It is thought that this linking causes the essence of energy treatment applied to the figurine to be transferred to the patient at the relevant locations in the energy field of the subject, represented by the treatment points on the figurine. The strongest linking is thought to occur from a subject holding the figurine or otherwise being in physical contact therewith. Linking also results from a subject and figurine being in proximity. This proximal linking is further enhanced when a subject is asleep or meditating. It is possible that the lack of conscious thought during sleep improves this proximal linking.

The potential applications of the present invention to many types of living bodies for a wide variety of physiologic and psychological purposes cannot presently be overstated. The development of these applications is merely a matter of using a generally appropriate figurine to repeatedly illuminate possible treatment points for a proximal subject over some period of time and observing the results. For example, different medical treatments unrelated to chi gong might be developed by use of the present invention. Also, domestic animal growth and productivity might be enhanced with the use of the present invention.

FIG. 7 shows a side sectional or schematic view of a figurine 80 constructed in accordance with another embodiment of the present invention. Figurine 80 is approximately sectioned along a midline of torso 82 and shows a plurality of energy conductors 84, 85, 86 located within figurine 80. Also included is a control device 88. Energy conductor 84 is approximately located along a portion of the Governing Vessel GV (FIG. 2), and energy conductor 85 is approximately located along a portion of the Conception Vessel CV (FIG. 1). Energy conductor 86 is generally located along a portion of the liver or kidney meridian or channel, which are known in acupuncture practice. Control device may be located as shown in the general area of Dantian 24 (FIG. 1), an energy storage vessel addressed in acupuncture practice. Also represented are a multiplicity of energy conversion devices 90, which may be useful in the present embodiment. While it is the purpose of conductors 84-86 to create an energy flow within figurine 80 to stimulate bodily energy flow in a subject, it may be even more beneficial to further localize energy concentration within figurine 80 with the use of conversion devices 90.

Energy conductors 84-86 and control device 88 may be adapted to cause energy flow upwardly through conductor 84 and downwardly through conductors 85 and 86. This path represents a nominal energy flow path known in mediation or chi gong practice, and thereby can be used to achieve the benefits of such practice without specific training for such practice. Alternatively, the conductors might be arranged and energized to cause energy flow in just a downward direction in figurine 80. An external connection (not shown) may be provided to the upper portion of figurine 80 for this purpose.

Whereas the embodiment of FIGS. 3 and 4 is described to include the use of electrical conductors and illumination devices, any suitable form of energy, energy conduction devices, and/or energy waveforms may be used in accordance with the embodiment of FIG. 7. Following are several examples. Other forms of electrical energy flow, like alternating current and even higher frequencies may be used within the figurine to create a healing effect. Such waveforms may be used in combination with inductive or capacitive devices or conductors located with the figurine. The inductive effects of simple electrical conductors may be used to achieve a beneficial healing effect either in combination with or without such conductive or capacitive devices. For example, control device 88 may take the form of an oscillating circuit or component for causing alternating current to flow through conductors 84-86. Depending upon oscillation frequency, the natural inductance of conductors 84-86 may be used in combination with a capacitive component within control device 88.

Alternatively, fiber optic conductors may be used for conductors 84-86 to provide an appropriate energy flow, and control device 88 may be adapted to deliver power and illumination in addition to an extensive control function. This approach may be combined with light outlet ports of any suitable form at specific energy treatment points to increase the effect. These are represented by energy conversion devices 90. Likewise, any form of energy, such as electrical, magnetic, electromagnetic, mechanical, radiant, or chemical, may be used. Examples include audible or inaudible sonic energy, visible or invisible radiation, heat flow and even the energy of a chemical reaction.

The waveforms generated for causing energy flow with any form of energy, may be varied to enhance the healing effect. For example, the waveform of the DC current used for illuminating the LEDs may be periodically interrupted to form a square wave and cause the LEDs to flash. Waveforms may be varied between different treatment points and between treatments for different conditions. An energy blockage in a subject might be more effectively cleared by a flashing waveform, while a low energy condition might be more effectively treated by a steady waveform. Likewise, the energy impedance characteristics of the material used to construct the figurine may be varied, depending upon the type of energy used, for enhancing linkage with a subject and the healing effect. Thus materials more conducive to either light or electromagnetic energy transmission might be selected.

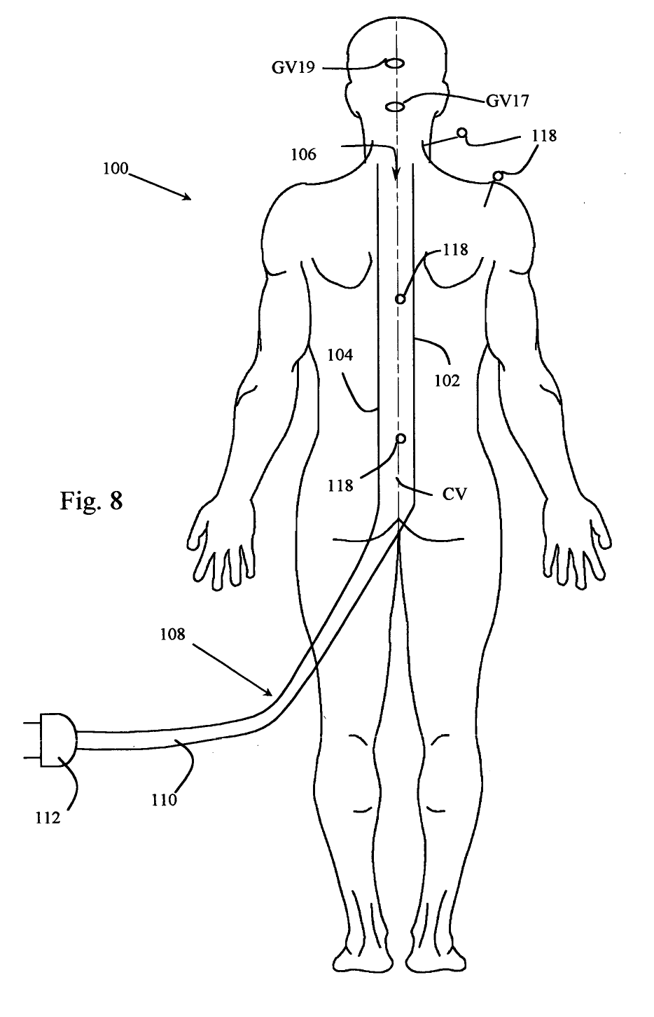

FIG. 8 shows yet another embodiment of the present invention, in which a figurine 100 has a pair of electrical conductors 102, 104 located on opposite sides of a spinal area 106. This general orientation is used to create an electromagnetic field between conductors 102, 104 and thus apply it across the spinal area 106 along a portion of the Governing Vessel GV. Conductors 102, 104 are intended to be energized by circuitry 108, which may be as simple as an AC power cord 110 and plug 112, which is adapted to access common power sources. The application of an AC voltage across conductors 102, 104 causes a repeated reversal of the electromagnetic field across spinal area 106 and thus causes electromagnetic stimulation of that area on figurine 100. The proximal location of figurine 100 to a treatment subject thus causes linking of this stimulation to the subject's energy field and provides the generic prophylactic treatment described above.

Conductors 102, 104 may be located either on the outside of or within figurine 100. Likewise, they may be located in front of and behind spinal area 106. To provide additional healing with figurine 100, the lower portion 114 of conductors 102, 104 maybe wrapped around legs 116L, 116R of figurine 100, for the purpose of treating various leg and joint conditions, as well as for stimulating a known chi gong energy removal function. In one embodiment conductors 102, 104 are formed with simple 300 ohm television antenna transmission line, in which a uniform spacing between conductors 102, 104 is maintained by insulation.

The performance of figurine 100 may be further enhanced by enabling the insertion of push-pins 118 into figurine 100. Steel push-pins, or acupuncture needles may act as small antennas for receiving and concentrating electromagnetic energy delivered by conductors 102, 104. In one embodiment, figurine 100 is provided as a commercially available, acupuncture training doll, which is specifically designed to allow needle insertion. Likewise, the above mentioned antenna transmission line may also be penetrated by push-pins, with appropriate care, to enhance energy concentration in spinal area 106. This may be done with appropriate shock hazard and shorting protection and may even include electrical contact with individual conductors 102, 104.

Circuitry 108 may alternatively include any form of control device (not shown) for performing any suitable control function, such as those described in reference to other embodiments herein. Circuitry 108 may likewise include a combination battery power supply and oscillator (not shown) for creating a portable device having the same healing effect.

The embodiments described above offer several advantages. They are much more effective for personal use than a laser pointer and doll because multiple treatment points can be addressed for more effective stimulation of healing energy flow. The programmable treatment modes further enhance the effectiveness of treatment by providing sound treatment protocols based upon clinical experience, which may be stored in a memory, or even hard wired, and thus not require learning by a user. These protocols may be activated without continual attention by a subject and even while a subject is asleep. Thus, the effectiveness of treatment is further enhanced by the improved linking which occurs during sleep. Subjects are also much more likely to perform treatment during sleep rather than while awake because of the significant time savings, thus further making treatment more effective.

In as much as acupuncture and other forms of energy healing have existed for so long and there have been and still are thousands of practitioners, the specific treatment points used for each ailment may be changed to suit the treatment points of different practitioners. The internal programming of control computer 65 (FIG. 5) may be changed to cause different treatment points to be used for each ailment. Likewise devices constructed in accordance with embodiments of the present invention may be programmed by energy healing practitioners for individual patients to enable more effective use between professional appointments.

The simple convenience of use of the present embodiments is also significant. Treatment points and methodologies do not need to be learned. Treatment is available both with and without close supervision of a practitioner. Treatments are much more conveniently accomplished during sleep periods. Laser and doll alignment does not need to be maintained. Treatment may easily be applied while a subject is traveling, by holding the figurine while sitting in a vehicle, for example. The savings of time afforded to individuals by performing treatment during periods of sleep and travel is very significant.

The present invention is illustratively described above in reference to the disclosed embodiments. Various modifications and changes may be made to the disclosed embodiments by persons skilled in the art without departing from the scope of the present invention as defined in the appended claims.

What is claimed it is:

1. An energy manipulation device, comprising:
a figurine substantially shaped like at least a portion of a living body;
one or more energy conduction elements located either on or within the figurine to apply an energy field to predetermined portions of the figurine corresponding to energy healing locations of the living body; and
circuitry adapted for energizing said one or more energy conduction elements.

2. The energy manipulation device of claim 1, further comprising separate illumination devices located at one or more of said predetermined portions of the figurine.

3. The energy manipulation device of claim 2, wherein said separate illumination devices are light emitting diodes or laser diodes.

4. The energy manipulation device of claim 2, wherein said circuitry is adapted to cause flashing of said separate illumination devices.

5. The energy manipulation device of claim 1, wherein said circuitry includes a programmable control device adapted for determining energizing of said one or more energy conduction elements.

6. The energy manipulation device of claim 5, wherein said control device is adapted to allow selection of a code corresponding to a particular ailment for causing activation of said energy conduction elements corresponding to treatment for said particular ailment.

7. The energy manipulation device of claim 6, wherein said control device is adapted to cause sequential treatment of a plurality of ailments based upon initial selection of the plurality of ailments.

8. The energy manipulation device of claim 5, wherein said control device is adapted to determine an energy waveform for energy flow along said one or more energy conduction elements.

9. The energy manipulation device of claim 1, wherein said conduction elements include electrical conductors located to create an electromagnetic field across said predetermined portions of the figurine.

10. The energy manipulation device of claim 9, wherein said electrical conductors include a pair of conductors generally located on opposing sides of said predetermined portions of the figurine.

11. The energy manipulation device of claim 10, wherein said pair of electrical conductors are generally located on opposing sides of a portion of said figurine representing a spine of the living body.

12. The energy manipulation device of claim 10, wherein said circuitry is adapted to apply an AC voltage across said pair of electrical conductors.

13. The energy manipulation device of claim 12, wherein said figurine is adapted to allow pins to pierce an outer surface thereof for concentrating electromagnetic energy at selectable points on said figurine.

14. The energy manipulation device of claim 1, wherein said energy conduction elements are routed through the figurine in accordance with portions of chi gong energy vessels or meridians of the living body.

15. The energy manipulation device of claim 1, wherein said figurine is miniaturized.

16. A method for performing energy healing on a subject, comprising the steps of:
locating a figurine representing at least a portion of a subject within proximity of the subject; and
routing artificial energy flow within the figurine at locations corresponding to energy healing locations within the subject.

17. The method of claim 15, wherein said step of routing includes conducting electrical or optical energy through the figurine.

18. The method of claim 15, further comprising the steps of repeating the steps of locating and routing with a specific subject over a period of time and observing any effects produced in the subject.

19. The method of claim 15, wherein said step of routing is adapted to enhance physical or mental performance of the subject.

20. The method of claim 15, wherein said figurine is substantially shaped like a human or animal subject.
